(19) United States
(12) Patent Application Publication
(10) Pub. No.: US 2021/0290666 A1
(43) Pub. Date: Sep. 23, 2021

(54) ANTIMICROBIAL and ANTI-INFLAMMATORY COMPOSITION

(71) Applicant: Howard J. Loff, Atlantic Beach, FL (US)

(72) Inventor: Howard J. Loff, Atlantic Beach, FL (US)

(21) Appl. No.: 17/341,812

(22) Filed: Jun. 8, 2021

(51) Int. Cl. A61K 33/18; A61K 31/4015; A61K 33/20; A61K 9/00

(52) U.S. Cl. CPC A61K 33/18; A61K 9/0048; A61K 33/20; A61K 31/4015

(57) ABSTRACT

Disclosed is a complex of povidone, iodine, and hypochlorous acid. The disclosed complex having a broad-spectrum antimicrobial and anti-inflammatory properties.

### FIELD OF INVENTION

The present invention relates to an antimicrobial composition, and more particularly, the present invention relates to a synergistic composition that is effective against a broad range of microorganisms.

### BACKGROUND

Hypochlorous acid is one of the most potent all-natural antimicrobial agents that is effective against a broad range of microorganisms. It is known to kill virtually all known pathogens and Prions. In proper concentrations, the hypochlorous acid is relatively non-toxic, non-irritant, and non-corrosive. Hypochlorous acid is naturally produced by the White Blood Cells in humans as an effective defense against invading pathogens. Hypochlorous acid is generally produced in a lab through electrolysis. However, hypochlorous acid is unstable and deteriorates rapidly. Thus, having a very short half-life.

Povidone-iodine (iodopovidone) is an iodophor solution containing a complex of triiodide and polyvinylpyrrolidone (Povidone, PVP). Povidone is a hydrophilic polymer widely employed as a carrier in the pharmaceutical, biomedical, and nutraceutical fields. The addition of Iodine forms a complex that binds the Iodine which helps stabilize the Iodine as well as results in a “slow” release of the Iodine to optimize its antimicrobial properties. Free iodine is slowly liberated from the complex and kills microorganisms. The Povidone-iodine is safer to use than a tincture of iodine that causes irritation, staining, and toxicity. As an iodophor, the iodine bind to povidone to form a complex, and the concentration of free iodine in the complex is insignificant.

A need is always there for more effective and safer antimicrobial compositions. A need is always there for antimicrobial compositions that are effective against a broader range of microorganisms and yet safer and non-irritant in humans. A need is always there for new antimicrobial compositions that are effective against harder microorganisms.

### SUMMARY OF THE INVENTION

The principal object of the present invention is therefore directed to a synergistic antimicrobial composition.

It is another object of the present invention that the composition is stable.

It is still another object of the present invention that composition has a long shelf life.

It is a further object of the present invention that the composition is effective against a broad range of microorganisms.

It is still a further object of the present invention that the composition is relatively non-irritant and non-toxic in humans.

It is an additional object of the present invention that the product has a broad range of applications.

It is yet another object of the present invention that the product is economical to manufacture.

In one aspect, disclosed herein is a composition that includes a complex of polyvinylpyrrolidone (povidone), iodine, and hypochlorous acid. The free iodine and hypochlorous acid's free available chlorine acid can be slowly released at the site of action.

In one aspect, the complex includes 0.02% hypochlorous acid and 0.05% Povidone-Iodine.

In one aspect, the complex includes 0.005% to 0.05% Hypochlorous Acid and 0.01% to 0.2% Povidone-Iodine.

These and other objects and advantages of the embodiments herein will become readily apparent from the following detailed description.

### DETAILED DESCRIPTION

Subject matter will now be described more fully hereinafter. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as assembly and methods of use thereof. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

Unless otherwise indicated, all numbers expressing quantities of ingredients used in this disclosure are to be understood as being modified in all instances by the term “about”. Accordingly, unless indicated to the contrary, the numerical parameters set forth in this disclosure are approximations that may vary depending upon the desired properties sought to be obtained by the present disclosure. At the very least, and not as an attempt to limit the application of the doctrine of equivalents to the scope of any claims, each numerical parameter should be construed in light of the number of significant digits and ordinary rounding approaches.

In general, the “effective amount” of an active agent or a pharmaceutical composition refers to the amount necessary to elicit the desired biological response. As will be appreciated by those of ordinary skill in this art, the effective amount of an agent may vary depending on such factors as the desired biological endpoint, the agent being delivered, the disease being treated, the subject being treated, etc. The “effective amount” may also vary from person to person and can be based on the age and weight of the person. The “effective amount” may also be based on the delivery route for the active agent, such as oral and parenteral.

“Pharmaceutical composition” or “compositions” or “therapeutic compositions” hereinafter refers to preparations which are in such a form as to permit the biological activity of the active agents to be unequivocally effective, and which contain no additional components which are toxic as administered to the patients.

Treatment refers to medical treatment and includes both therapeutic and prophylactic treatment depending on the context of use herein. “Treatment” includes preventing or reducing the complications, and symptomatic relief.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

Disclosed herein is an antimicrobial composition that is effective against a broad range of microorganisms. The disclosed composition is synergistic that is effective against a broader range of microorganisms compared to its components. The disclosed composition includes a complex of povidone, iodine, and hypochlorous acid. The povidone acts as a carrier molecule which slowly releases the free iodine and hypochlorous acid on contact with human tissue for antimicrobial and anti-inflammatory activity.

The hypochlorous acid and iodine act through different mechanisms of action. Thus, the combined iodine and hypochlorous acid are more effective against a broad range of microorganisms. Moreover, the combination was found to be synergistic. Although the povidone-iodine complex slowly releases free iodine at the site of action, which minimizes the toxicity of the iodine, however, the povidone-iodine complex and the free iodine are still irritant, which limits its application. The use of povidone-iodine in bigger wounds is generally not advisable because the higher amounts of the povidone-iodine complex may cause toxicity. The disclosed complex of povidone, iodine, and hypochlorous acid requires lesser loading of iodine. Thus, the disclosed composition is relatively non-toxic and non-irritant than convention povidone-iodine complex, while effective against a broader range of microorganism and having multiple applications.

The hypochlorous acid is unstable and fast loses its potency. The complex of hypochlorous with povidone and iodine was found to be stable and have a long shelf life. The hypochlorous acid in the disclosed complex is stable and slowly released at the site of action for killing the microorganisms. The disclosed complex of povidone, iodine, and hypochlorous acid is synergistically potent and stable. Povidone, a carrier molecule, binds the hypochlorous acid, thereby resulting in the significant stabilization of hypochlorous acid and increases its shelf life.

Besides having superior antimicrobial activity, the disclosed composition having a broad range of applications in the healthcare industry, sanitization, disinfectant, food processing and handling. Specifically, in the medical field, the disclosed composition can have broad applications including potent topical antimicrobial for treating infections and disinfections in preoperative surgical preparations. Additionally, the disclosed composition has been found to have potent anti-inflammatory properties which significantly aid in tissue healing and provides symptomatic relief. The disclosed composition can also be used as an eye drop for treating eye infections. Moreover, the disclosed composition, in proper concentration, can be an effective preservative for eye formulations, such as drops, ointment, or gels.

The disclosed complex is a broad-spectrum antimicrobial for an eye infection and additionally, anti-inflammatory properties make the disclosed composition a long-needed and potentially a new therapy for the treatment of eye infections.

### Example 1: Preparation of a Complex Having Povidone, Iodine, and Hypochlorous

80 ml of Hypochlorous Acid in a concentration of 0.025% was added slowly with mixing to 1 ml of solution containing 5% povidone-iodine (0.5% available iodine) and thereafter 19m1 of normal saline was added. The mixture was slowly mixed till a homogenous solution was obtained.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. An antimicrobial and anti-inflammatory composition comprising:
a complex of povidone, iodine, and hypochlorous acid.

2. The antimicrobial composition according to claim 1, wherein the povidone is a carrier molecule that slowly releases the iodine and active chlorine from the hypochlorous acid.

3. A method for treating eye infections, the method comprising:
administering a composition comprising povidone, iodine, and hypochlorous acid,
wherein the iodine and active chlorine from the hypochlorous acid are slowly released from the povidone into the eye.

4. The method according to claim 3, wherein the composition is present in the form of eye drops.

5. The method according to claim 3, wherein the composition is present in the form of an ointment or gel.

6. A process of manufacturing an eye formulation, the method comprising:
providing an eye formulation; and
adding a complex of povidone, iodine, and hypochlorous acid in the eye formulation as a preservative in a predetermined preservative concentration.

7. The process according to claim 6, wherein the eye formulation is eye drops.

8. The process according to claim 6, wherein the eye formulation is an ointment or gel.
